(19) United States
(12) Plant Patent Application Publication
(10) Pub. No.: US 2015/0020282 P1
(43) Pub. Date: Jan. 15, 2015

(54) Sutera plant named 'KLESG13255'

(50) ;

(71) Applicant: Klemm+Sohn GmbH & Co. KG, Stuttgart (DE)

(72) Inventors: Nils Klemm, Stuttgart (DE); Katinka Wilde, Stuttgart (DE)

(21) Appl. No.: 13/987,190

(22) Filed: Jul. 9, 2013

(51) Int. Cl. A01H 5/00

(52) U.S. Cl. PLT/485

(57) ABSTRACT

A new Sutera plant particularly distinguished by having a white flower color, large flowers and more branching, is disclosed.

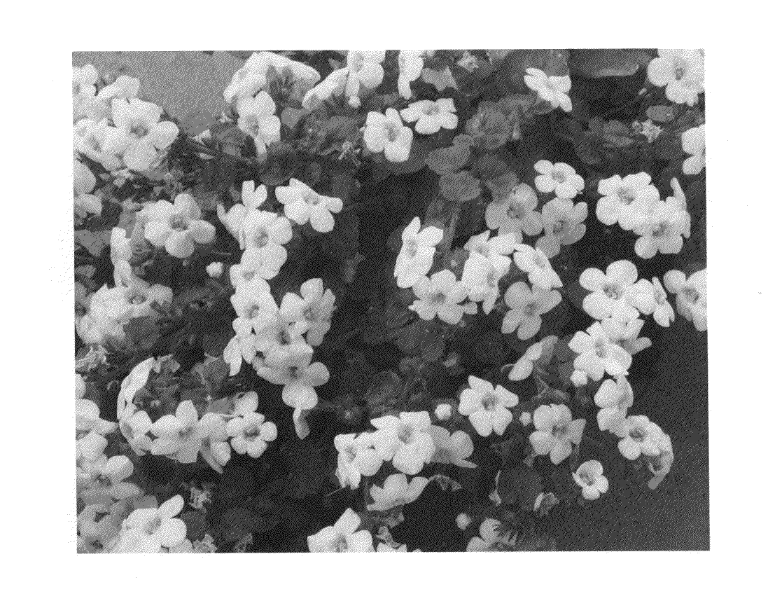

### GENUS AND SPECIES

Sutera grandiflora

### VARIETY DENOMINATION

‘KLESG13255’

### BACKGROUND OF THE NEW PLANT

The present invention comprises a new and distinct variety of Sutera, botanically known as Sutera grandiflora, and hereinafter referred to by the variety name ‘KLESG13255’. ‘KLESG13255’ originated from an open-pollination between the proprietary female Sutera variety ‘G Lavender’ (unpatented) and an unknown male Sutera variety.

In August of 2009, seed was collected from the open-pollination cross of two Sutera parental lines, ‘G Lavender’ and an unknown Sutera in Stuttgart, Germany. The seeds were sown and plants were grown for evaluation. A plant line was selected in May 2010, which had a white flower color and subsequently named ‘KLESG13255’. In May 2010, ‘KLESG13255’ was first vegetatively propagated by in-vitro cultivation. ‘KLESG13255’ was found to reproduce true to type in successive generations of asexual propagation via in-vitro cultivation.

### CROSS REFERENCE TO RELATED APPLICATIONS

Plant Breeder's Rights for ‘KLESG13255’ were applied for in Canada on Feb. 8, 2013, Application No. 13-7928. ‘KLESG13255’ has not been made publicly available or sold more than one year prior to the filing date of this application.

### SUMMARY

The following are the most outstanding and distinguishing characteristics of this new variety when grown under normal horticultural practices in Stuttgart, Germany.

1. White flower color;

2. Large flowers; and

3. More branching.

### DESCRIPTION OF THE PHOTOGRAPH

This new Sutera plant is illustrated by the accompanying photograph which shows a close-up of the flowers. The photograph is of a 10-week-old plant grown in a controlled greenhouse environment in Stuttgart, Germany in May 2012. The colors shown are as true as can be reasonably obtained by conventional photographic procedures.

### DESCRIPTION OF THE NEW VARIETY

The following detailed descriptions set forth the distinctive characteristics of ‘KLESG13255’. The data which define these characteristics were collected from asexual reproductions carried out in Stuttgart, Germany. Data was collected on 11-week-old plants grown in a controlled greenhouse environment in Stuttgart, Germany in May 2013. Color references are to The R.H.S. Colour Chart of The Royal Horticultural Society of London (R.H.S.), (2005).
Classification:
   Family.—Scrophulariaceae.
   Botanical.—Sutera grandiflora.
   Common.—Sutera Designation: ‘KLESG13255’.
Parentage:
   Female parent.—Proprietary Sutera line ‘G Lavender’ (unpatented).
   Male parent.—An unknown Sutera line.
Plant:
   Habit.—Trailing.
   Height.—About 9.0 cm.
   Spread.—37.0 cm.
   Life cycle.—Blooms from April until September.
   Form.—Round.
   Time to produce a rooted cutting.—4 weeks.
   Time to bloom from propagation.—5 weeks.
   Lastingness of inflorescence on the plant.—About 7 days.
Stems:
   Number of stems per plant.—9.
   Length.—About 23.0 cm.
   Diameter.—0.8 cm.
   Internode length.—About 1.5 cm.
   Color.—RHS 144A.
   Texture.—Smooth.
   Anthocyanin.—Absent.
Leaves:
   Arrangement.—Single, opposite.
   Shape.—Ovate, acute angle to the stem.
   Apex.—Acute.
   Base.—Cordate.
   Margin.—Serrate.
   Length.—Between 1.6 cm to 2.5 cm.
   Width.—Between 1.8 cm and 3.0 cm.
   Color.—Immature and mature leaves: Upper surface: RHS 137A Lower surface: RHS 137C.
   Texture.—Both upper and lower surfaces: Leathery.
   Venation type.—Pinnate.
   Venation color.—Upper and lower surfaces: RHS 144A.
   Fragrance.—Present.
Inflorescence:
   Blooming habit.—Freely flowering.
   Quantity of inflorescence per stem.—About 10.
   Type of inflorescence.—Flowers are often solitary, most often 2 flowers per node are developed.
   Fragrance.—Present.
Pedicel:
   Color.—RHS 144A.
   Length.—1.0 cm to 1.5 cm.
   Diameter.—About 0.1 cm.
   Texture.—Pubescent.
Bud:
   Shape.—Tubular.
   Diameter.—About 0.3 cm.
   Length.—About 0.5 cm.
   Color (at tight bud).—RHS 155A.
Corolla (flower):
   Type and shape.—Single-type, nearly round, lower part tube and funnel shaped formed by 5 fused petals, upper part is salver shaped formed by the partly free petal lobes.
   Corolla, horizontal diameter.—About 2.2 cm.
   Corolla, total length.—About 2.5 cm. Color of throat: Inner surface: RHS 23A Outer surface: RHS 15B Diameter: 0.4 cm Texture: Inner and outer surfaces: Smooth
Petal (lobes):
   Apex.—Rounded tips.
   Base.—Fused.
   Margin.—Entire.
   Color.—Inner surface: RHS N999D Outer surface: RHS N999D.
   Texture.—Upper and lower surfaces: Smooth.
Calyx:
   Arrangement and appearance.—Whorl of 5 sepals surrounding the base of the flower tube, sepals are fused at the base.
   Sepals.—Color: Upper and lower surfaces: RHS 139C Length: 1.2 cm Width: 0.1 cm Apex: Acute Base: Fused Margin: Entire Texture: Upper and lower surfaces: Densely pubescent.
Reproductive organs:
   Stamens.—Quantity: 4 Filament: Color: RHS 155B Length: 1.1 cm Diameter: 0.1 cm Anther color: RHS 15D Pollen amount: Abundant Pollen color: RHS 22A.
   Pistil.—Quantity per flower: 1 Length: 1.3 cm Stigma color: RHS 145C Style color: RHS 155B.
Fruit and seed set:
   Capsulates/fruit.—Color: RHS 137C Size: 0.5 cm×0.4 cm. Shape: Ovate.
   Seeds.—Color: RHS 20A Size: About 0.1 cm
   Shape.—Round.
Disease and pest/insect resistance: None observed.

### COMPARISON WITH PARENTAL LINE AND KNOWN VARIETY

‘KLESG13255’ is a distinct variety of Sutera having a white flower color; ‘KLESG13255’ is distinguished from its female parent as described in Table 1:

TABLE 1
Comparison with Parental Line

| | Variety | Female parent |
|---|---|---|
| Trait | ‘KLESG13255’ | ‘G Lavender’ |
| Color of flower | White | Lavender |

‘KLESG13255’ is most similar to the Sutera plant named ‘Summer White Falls’ (unpatented). Differences between the two varieties are described in Table 2:

TABLE 2
Comparison with Similar Variety

| | | Commercial line |
|---|---|---|
| | Variety | ‘Summer White Falls’ |
| Trait | ‘KLESG13255’ | (unpatented) |
| Flower | Bigger flowers than ‘Summer | Smaller flowers than |
| size | White Falls’ | ‘KLESG13255’ |
| Branching | More branching than ‘Summer | Less branching than |
| | White Falls’ | ‘KLESG13255’ |

We claim:

1. A new and distinct variety of Sutera plant named ‘KLESG13255’ as shown and described herein.
